(19) United States
(12) Patent Application Publication
(10) Pub. No.: US 2026/0224557 A1
(43) Pub. Date: Aug. 6, 2026

(54) NRF2 ACTIVATOR

(71) Applicant: Ivy Cosmetics Co., Ltd., Tokyo (JP)

(72) Inventors: Masatoshi TAKEIRI, Tokyo (JP); Keiko ARAI, Tokyo (JP); Yoshihide KIMURA, Tokyo (JP)

(73) Assignee: Ivy Cosmetics Co., Ltd., Tokyo (JP)

(21) Appl. No.: 19/149,336

(22) PCT Filed: Nov. 25, 2024
(86) PCT No.: PCT/JP2024/041599

§ 371 Date: Jul. 18, 2025

(30) Foreign Application Priority Data
Sep. 28, 2023 (JP) 2023-167752

(51) Int. Cl. A61K 31/4745; A61K 9/00; A61K 9/06; A61K 9/20; A61K 36/24; A61K 47/14; A61K 47/20; A61K 47/26; A61K 47/44; A61K 47/46

(52) U.S. Cl. CPC A61K 31/4745; A61K 9/0056; A61K 9/06; A61K 9/2018; A61K 47/14; A61K 47/20; A61K 47/26; A61K 47/44; A61K 47/46; A61K 36/24

(57) ABSTRACT

An object of the present invention is to provide an Nrf2 activator for preventing or treating a disease in which oxidative stress involves in development thereof or progress of a symptom thereof. The Nrf2 activator of the present invention as a solution thereof contains a bisindole alkaloid represented by the general formula (1) below or a pharmaceutically acceptable salt thereof as an active ingredient.

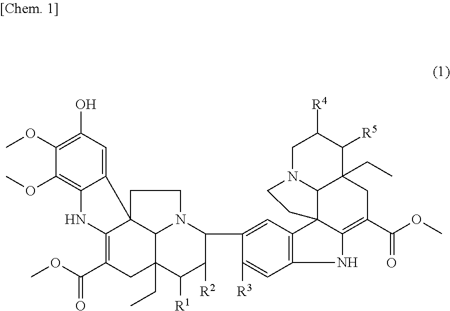

[In the formula, R¹ represents a hydroxy group or —O— together with R². R² represents —O— together with R¹ or —O— together with R³. R³ represents —O— together with R² or a hydrogen atom. R⁴ and R⁵ both represent a hydroxy group or together represent —O— or a bond.]

### TECHNICAL FIELD

The present invention relates to an Nrf2 activator.

### BACKGROUND ART

It is known that a transcription factor, Nrf2 (NF-E2 related factor 2), performs a function of protecting cells through increase in the expression of a gene for relieving oxidative stress or the like, as a type of protection mechanism against oxidative stress in the body. Thus, components for preventing or treating a disease in which oxidative stress involves in development thereof or progress of a symptom thereof through activation of Nrf2 have been recently searched intensively, and for example, Patent Document 1 discloses that an extract of Lycium fruit has an Nrf2-activating action. However, it is still significantly important to search for components having an Nrf2-activating action.

### PRIOR ART DOCUMENTS

### Patent Document

Patent Document 1: JP2012-211107A

### SUMMARY OF THE INVENTION

### Problems that the Invention is to Solve

Thus, an object of the present invention is to provide an Nrf2 activator for preventing or treating a disease in which oxidative stress involves in development thereof or progress of a symptom thereof.

### Means for Solving the Problems

As a result of intensive studies in view of the above points, the present inventors have found that conophylline represented by the chemical formula below, which is known as a bisindole alkaloid, has an Nrf2-activating action.

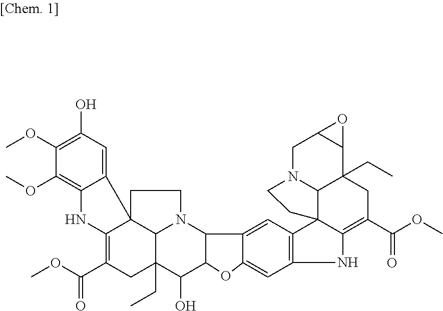

The Nrf2 activator of the present invention which has been made based on the above findings contains a bisindole alkaloid represented by the general formula (1) below or a pharmaceutically acceptable salt thereof as an active ingredient, as stated in claim 1.

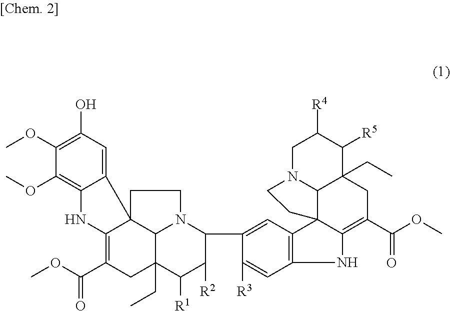

[In the formula, R¹ represents a hydroxy group or —O— together with R². R² represents —O— together with R¹ or —O— together with R³. R³ represents —O— together with R² or a hydrogen atom. R⁴ and R⁵ both represent a hydroxy group or together represent —O— or a bond.]

### Effect of the Invention

According to the present invention, an Nrf2 activator for preventing or treating a disease in which oxidative stress involves in development thereof or progress of a symptom thereof can be provided.

### BRIEF DESCRIPTION OF THE DRAWINGS

FIG. 1 A graph showing the Nrf2-activating action of conophylline in Examples.

### MODE FOR CARRYING OUT THE INVENTION

The Nrf2 activator of the present invention contains a bisindole alkaloid represented by the general formula (1) below or a pharmaceutically acceptable salt thereof as an active ingredient.

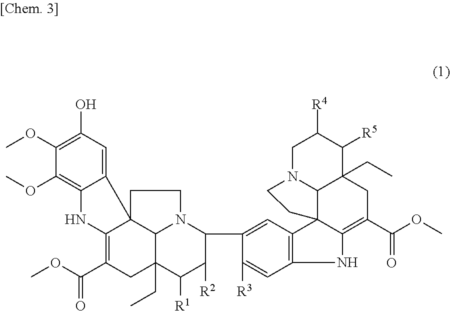

[In the formula, R¹ represents a hydroxy group or —O— together with R². R² represents —O— together with R¹ or —O— together with R³. R³ represents —O— together with R² or a hydrogen atom. R⁴ and R⁵ both represent a hydroxy group or together represent —O— or a bond.]

The pharmaceutically acceptable salt of the bisindole alkaloid represented by the general formula (1) above may be an inorganic acid salt (hydrochloride, sulfate, nitrate, phosphate, or the like), an organic acid salt (acetate, lactate, citrate, tartrate, maleate, fumarate, oxalate, or the like), or the like.

Specific examples of the bisindole alkaloid represented by the general formula (1) above include conophylline (R¹ is a hydroxy group, R² and R³ are together —O—, and R⁴ and R⁵ are together —O—), conophyllidine (R¹ is a hydroxy group, R² and R³ are together —O—, and R⁴ and R⁵ are together a bond), conofoline (R¹ and R² are together —O—, R³ is a hydrogen atom, and R⁴ and R⁵ are together —O—), conophyllinine (R¹ is a hydroxy group, R² and R³ are together —O—, and R⁴ and R⁵ are both a hydroxy group), and the like. These compounds are known as plant-derived bisindole alkaloids having an antitumor action or the like and can be isolated and purified from a plant of the Apocynaceae, such as Ervatamia microphylla and Tabernaemontana divaricata (when necessary, refer to JP4696303B, for example). These compounds used as an active ingredient of the Nrf2 activator of the present invention, however, are not limited to isolated and purified compounds and may be in the form of a solvent extract or the like containing such a compound. Moreover, these compounds may be synthesized by an organic chemistry method and may be stereoisomers (enantiomers and diastereomers) or racemates based on the presence of asymmetric carbon. Moreover, these compounds may be mixtures of two or more compounds and may be in the form of a solvent extract or the like containing two or more compounds. Here, the “solvent extract” means an extract of, for example, the above plants extracted using water or an organic solvent (alcohols such as monoalcohols exemplified by methanol, ethanol, and isopropanol and polyhydric alcohols exemplified by 1,3-butylene glycol, ether, hexane, ethyl acetate, acetonitrile, and the like, which may contain water) as the solvent for extraction.

The Nrf2 activator of the present invention itself can be taken or ingested as a medicine, a health food, or the like by formulating into various dosage forms, and the Nrf2 activator can also be blended into various foods and drinks and ingested. Moreover, the Nrf2 activator of the present invention can also be applied to the skin in the form of external preparation such as ointments, creams, and lotions. The dose, the intake, or the application amount thereof can be appropriately determined based on the age, the gender, the body weight, the degree of symptom, or the like of the subject of application, and by taking, ingesting or applying an adequate amount, a prophylactic effect or a therapeutic effect on a disease in which oxidative stress involves in development thereof or progress of a symptom thereof such as Alzheimer's disease, chronic obstructive pulmonary disease, asthma, atherosclerosis, multiple sclerosis, rheumatoid arthritis, and a disorder or aging of the skin due to light (including skin-beautifying effect and skin-lightening effect) is expected based on the Nrf2-activating action.

### EXAMPLES

Hereinafter, the present invention is explained in detail with Examples, but the present invention should not be construed as being limited to the following description.

### Experimental Example 1: Nrf2-Activating Action of Conophylline

### (Experimental Method)

The Nrf2-activating action of conophylline (crystal) which was isolated and purified from Ervatamia microphylla, which is a plant of the Apocynaceae, by the method described in JP4696303B was evaluated by ARE (Antioxidant Response Element)-luciferase reporter assay, which is known as a monitoring system for the Nrf2 antioxidant pathway. Specifically, a plasmid was obtained by inserting a polynucleotide having a sequence in which ARE sequence was repeated twice (TAGCTTGGAAATGACATTGCTAATGGTGACAAAGCAACTTTTAGCTTGGAAATGACA TTGCTAATGGTGACAAAGCAACTTT: SEQ ID NO: 1) into the multiple cloning site of pNL2.2 [NlucP/Hygro] Vector (Promega Corporation), which is a vector for luciferase reporter assay, and the plasmid was introduced into HaCaT cells (DKFZ) using Cell Line Nucleofector Kit V (Lonza) by electroporation. After 24 hours, by adding hygromycin at a final concentration of 600 μg/mL and selecting drug-resistant cells, an ARE-luciferase reporter-stably-expressing HaCaT cell line having hygromycin resistance was established. In a 96-well plate, about 10000 cells per well of the ARE-luciferase reporter-stably-expressing HaCaT cell line were seeded using 100 μL of a culture medium (high glucose DMEM (Sigma-Aldrich Co. LLC) containing 10% FBS (Gibco) and 200 mmol/L L-glutamine solution (×100) (FUJIFILM Wako Pure Chemical Corporation), the same applies below), and the cells were cultured for 16 to 24 hours in an incubator at 37° C. and 5% CO₂. Then, the culture medium was replaced with culture mediums obtained by adding conophylline at various final concentrations dissolved in DMSO (final concentration of DMSO of 0.1%), and the cells were further cultured for 24 hours. After 24 hours, the luciferase activities were measured using Nano-Glo Luciferase Assay System (Promega Corporation) according to the protocol, and thus the Nrf2-activating action of conophylline was evaluated.

### (Experimental Results)

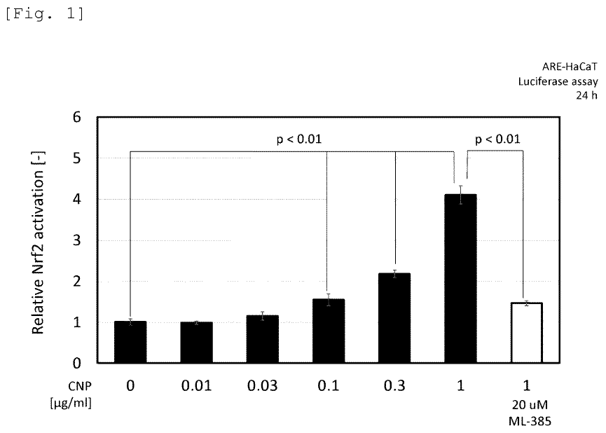

The relative values are shown in FIG. 1, where the luciferase activity of a case in which DMSO at a final concentration of 0.1% alone was added as a control is regarded as 1 (CNP represents conophylline). As it is obvious from FIG. 1, conophylline enhanced the luciferase activity in a concentration-dependent manner. ML-385 (Cayman Chemical Company), which is an Nrf2 inhibitor, at a final concentration of 20 μM dissolved in DMSO was added together (final concentration of DMSO of 0.1%) when conophylline at a final concentration of 1 μg/mL dissolved in DMSO was added, so that the luciferase activity decreased considerably. From the above results, the Nrf2-activating action of conophylline could be observed.

### Preparation Example 1: Tablet

A Tablet having the composition below and having an Nrf2-activating action was produced by a known method (unit: weight %).
Conophylline 1
Lactose 80
Magnesium stearate 19

### Preparation Example 2: Biscuit

A biscuit having the composition below and having an Nrf2-activating action was produced by a known method (unit: weight %).
Conophylline 1
Cake flour 32
Whole egg 16
Butter 16
Sugar 24
Water 10
Baking powder 1

### Preparation Example 3: Jelly

A jelly having the composition below and having an Nrf2-activating action was produced by a known method (unit: weight %).
Conophylline 0.01
Gelling agent 1.3
Sugar 20
pH-Adjusting agent 2.5
Water 76.19

### Preparation Example 4: Ointment

An ointment having the composition below and having an Nrf2-activating action was produced by a known method (unit: weight %).
Conophylline 5
White petrolatum 95

### Preparation Example 5: Cream

A cream having the composition below and having an Nrf2-activating action was produced by a known method (unit: weight %).
Conophylline 5
White petrolatum 30
Cetanol 15
Sodium lauryl sulfate 1
Ethyl parahydroxybenzoate 0.1
Butyl parahydroxybenzoate 0.1
Water 48.8

### Preparation Example 6: Lotion

A lotion having the composition below and having an Nrf2-activating action was produced by a known method (unit: weight %).
Conophylline 10
Polyoxyethylene (40) hydrogenated castor oil 0.5
Ethanol 5
Paraben 0.2
Water 84.3

### INDUSTRIAL APPLICABILITY

The present invention is industrially applicable because the present invention can provide an Nrf2 activator for preventing or treating a disease in which oxidative stress involves in development thereof or progress of a symptom thereof.

1: A method of activating Nrf2 for preventing or treating a disease in which oxidative stress involves in development thereof or progress of a symptom thereof, comprising the step of:
administering to a human a bisindole alkaloid represented by the general formula (1) below or a pharmaceutically acceptable salt thereof as an active ingredient 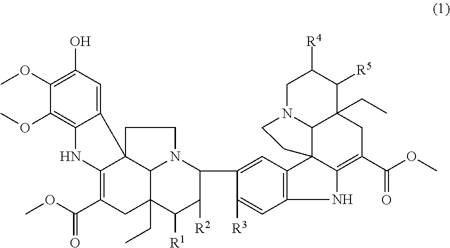
wherein, R¹ represents a hydroxy group or —O— together with R², R² represents —O— together with R¹ or —O— together with R³, R³ represents —O— together with R² or a hydrogen atom, R⁴ and R⁵ both represent a hydroxy group or together represent —O— or a bond.

2: The method of claim 1, wherein the active ingredient is administered at a dosage that has a preventing effect on said disease in said human.

3: The method of claim 1, wherein the active ingredient is administered at a dosage that has a treating effect on said disease in said human.

4: The method of claim 1, wherein the active ingredient is administered orally.

5: The method of claim 1, wherein the active ingredient is administered externally.


SEQUENCE LISTING (1 sequence)

SEQ ID NO: 1 - DNA, 82 bp, synthetic construct
  source 1..82: mol_type = other DNA
tagcttggaaatgacattgctaatggtgacaaagcaacttttagcttggaaatgacattgctaatggtgacaaagcaacttt